(19) United States
(12) Patent Application Publication
(10) Pub. No.: US 2016/0051723 A1
(43) Pub. Date: Feb. 25, 2016

(54) BIORESORBABLE TISSUE REPAIR COMPOSITION

(71) Applicants: Gregory J. Pomrink, Newberry, FL (US); Zehra Tosun, Gainesville, FL (US); Cecilia Cao, Gainesville, FL (US); Annabelle Woodruff, Gainesville, FL (US)

(72) Inventors: Gregory J. Pomrink, Newberry, FL (US); Zehra Tosun, Gainesville, FL (US); Cecilia Cao, Gainesville, FL (US); Annabelle Woodruff, Gainesville, FL (US)

(21) Appl. No.: 14/830,155

(22) Filed: Aug. 19, 2015

Related U.S. Application Data
(60) Provisional application No. 62/040,108, filed on Aug. 21, 2014.

(51) Int. Cl. A61L 26/00; A61L 24/04

(52) U.S. Cl. CPC A61L 26/009; A61L 24/043; A61L 26/0004; A61L 2300/236; A61L 2300/412

(57) ABSTRACT

Compositions including hyaluronic acid or derivative thereof, a borate containing crosslinking agent, a di or polyvalent metal ion, and, optionally, one or more of N-hydroxysuccinimide, and/or a cationic monomer, oligomer, or polymer selected from the group consisting of hydroxylysine, poly(N-methylethylamine), ε-poly-lysine, or polyamine, or a combination thereof are described. Also, methods for making a bioresorbable tissue repair composition and methods of correcting, sealing, connecting or repairing tissue by contacting the tissue with the bioresorbable tissue repair composition are described.

### CROSS-REFERENCE TO RELATED APPLICATIONS

This application claims the benefit of U.S. Provisional Patent Application No. 62/040,108, filed Aug. 21, 2014, the entire contents of which are hereby incorporated herein by reference.

### BACKGROUND

A bioresorbable tissue repair composition is disclosed herein. The compositions are useful for medical or veterinary use in repair of physical damage to hard and soft mammalian tissues such as cuts, tears, holes, bone breaks and other injuries/defects resulting from surgery or trauma.

One commonly used tissue sealant is fibrin glue, a material analogous to clotted blood, which is obtained from reaction of fibrinogen and thrombin isolated from blood plasma. For example, see “Fibrin Glue from Stored Human Plasma: An Inexpensive and Efficient Method for Local Blood Bank Preparation,” William D. Spotnitz, M. D., Paul D. Mintz, M. D., Nancy Avery, M. T., Thomas C. Bithell, M. D., Sanjiv Kaul, M. D., Stanton P. Nolan, M. D. (1987), The American Surgeon, 53, 460-62. However, concern about possible viral or prion contamination of human blood-derived protein products, and dissatisfaction with the relatively long times often required for fibrin gelation or “setting” to occur, have resulted in a search for tissue sealants with more advantageous properties. There have been systems developed that use fibrin glues as part of a more complex assembly with more favorable properties. U.S. Pat. No. 6,699,484 discusses the use of fibrinogen in mixtures with polysaccharides such as hyaluronan and chitosan to form surgical adhesives. The fibrinogen and thrombin components are distributed in dry form on a support comprising the polysaccharide, which is activated by water when placed on a wound to form a sealant.

A tissue sealant that does not use proteins isolated from mammalian blood, such as Duraseal® produced by Confluent Surgical Inc. of Waltham, Mass., comprises tri-lysine-amine and an activated polyethyleneglycol. A similar product, termed CoSeal® and produced by Baxter of Deerfield, Ill., is likewise composed of synthetic functionalized polyethyleneglycol derivatives, also avoiding the use of blood-derived materials. However, both of these synthetic hydrogels are dimensionally unstable in the presence of water, undergoing considerable swelling. For example, see “Evaluation of Absorbable Surgical Sealants: In vitro Testing,” Patrick K. Campbell, PhD, Steven L. Bennett, PhD, Art Driscoll, and Amar S. Sawhney, PhD, at www.duralsealant.com/duralsealant/literature.htm (as of Aug. 24, 2006). This tendency to swell can be highly disadvantageous in certain applications, such as neurosurgery, where the resulting pressure on nerve or brain tissue can produce serious side-effects.

Published PCT application WO2005/113608 and Published U.S. patent application no. 2005/0271729 discuss the crosslinking of chitosan and hyaluronan, also known as hyaluronic acid. Hyaluronan is an acidic linear polysaccharide formed of /3-1,3 linked dimeric units, the dimeric units consisting of an 2-acetamido-2-deoxyglucose and D-gluconic acid linked in a /3-1,4 configuration. These published applications discuss crosslinking the two types of polysaccharides using a carbodiimide reagent.

U.S. Pat. No. 6,703,444 (the '444 patent) discloses a process for the production of hyaluronic acid derivatives including cross-linked hyaluronic acid/polyvinyl alcohol. In particular, the process relates to multiple cross-linked hyaluronic acid derivatives, to cross-linked derivatives so obtained, and to products containing them and their uses in cosmetic, medical and pharmaceutical applications. Synthetic polymers disclosed include polyvinyl alcohol (PVA), polyethylene oxide (PEO), and polypropylene oxide (PPO), as well as copolymers of any of the aforementioned polymers, polyacrylic acid, polyacrylamide and other hydroxyl, carboxyl and hydrophilic synthetic polymers.

U.S. Pat. No. 6,903,199 (the '199 patent) discloses water-insoluble, crosslinked amide derivatives of hyaluronic acid and manufacturing method thereof, where the amide derivatives of hyaluronic acid are characterized by crosslinking, of polymer or oligomer having two or more amine groups, with hyaluronic acid or its hyaluronate salts through an amidation reaction. The water-insoluble, crosslinked amide derivatives of hyaluronic acid are disclosed as diversely used for prevention of adhesion after surgical operation, correction of facial wrinkles, dermal augmentation, tissue engineering, and osteoarthritic viscosupplement. However, the compositions described in the '199 patent are described as water insoluble, and have “ . . . overcome demerit of existing HA derivatives to be easily decomposed in the living body . . . ”. The materials described in the '199 patent are therefore not readily bioresorbable.

### SUMMARY

Disclosed herein is a composition comprising: (1) hyaluronic acid or derivative thereof, (2) a borate containing crosslinking agent, (3) a metal containing solvent, and (4) optionally one or more monomer, oligomer, or polymer selected from the group consisting of hydroxylysine, poly(N-methylethylamine), ε-poly-lysine, polyethylenimine (PEI), diethylenetriamine (DETA), or triethylenetetramine (TETA). Methods of preparing such compositions and their use in tissue repair are also disclosed.

### DETAILED DESCRIPTION OF THE DRAWINGS AND THE PRESENTLY PREFERRED EMBODIMENTS

There is a need for a tissue repair composition that: (1) is not blood or animal protein derived, (2) may include other biocompatible materials, (3) is dimensionally stable after placement in the patient's body, (4) is bioresorbable, (5) has good sealant, tissue adhesive and endothelial cell attachment properties, (5) is of sufficient strength and elasticity to effectively seal biological tissues. It is further desirable for such a composition to be readily prepared and used during surgery to form a tissue seal on a timescale compatible with surgery on living patients.

The composition of the present invention includes: (1) hyaluronic acid or derivative thereof, (2) a borate containing crosslinking agent, (3) a di or polyvalent metal ion, and (4) optionally one or more cationic monomer, oligomer, or polymer selected from the group consisting of hydroxylysine, poly(N-methylethylamine), ε-poly-lysine, polyethylenimine, or polyamine.

The composition uses hyaluronic acid which is a naturally occurring polymer associated with various cellular processes involved in wound healing, such as angiogenesis. Hyaluronic acid also presents unique advantages: it is easy to produce and modify, hydrophilic and naturally biodegradable. The composition disclosed herein provides materials for medical or veterinary use in repair of physical damage to hard and soft mammalian tissues such as cuts, tears, holes, bone breaks and other injuries/defects resulting from surgery or trauma. It further provides a non-thrombogenic surface that promotes normal endothelization compared to synthetic biomedical polymers that do not support endothelial cell attachment and proliferation.

In one embodiment, the crosslinking agent is one or more of boric acid, sodium borate, sodium tetraborate, disodium tetraborate, sodium tetraborate decahydrate, anhydrous borax (Na2B4O7), borax pentahydrate (Na2B4O7.5H2O), borax decahydrate (Na2B4O7.10H₂O), sodium borohydride, tributyl borate, triethanolamine borate, tris(trimethylsilyl)borate, tris-borate-EDTA buffer, triethyl borate, triisopropyl borate, trimethyl borate or another organoborate.

In another embodiment, a method for making a bioresorbable tissue repair composition is disclosed comprising (1) mixing (a) hyaluronic acid or derivative thereof, (b) a borate containing crosslinking agent, (c) a di or polyvalent metal ion, and (d) optionally one or more monomer, oligomer, or polymer selected from the group consisting of hydroxylysine, poly(N-methylethylamine), ε-poly-lysine, polyethylenimine, or polyamine, and (2) lyophilizing or drying the mixture.

The mixture may then be lyophilized to form a sponge. The sponge may also be dehydrothermally treated (DHT) to dehydrothermally treated (DHT) to further induce crosslinking between the carboxylic acid and hydroxyl groups to form ester within and between chains of the polysaccharide along with forming amide from the condensation of carboxylic acid and primary amine moieties.

In another embodiment a material possessing a 3d porous structural composite is prepared by mixing hyaluronic acid or a derivative thereof with one or more monomer, oligomer, or polymer selected from the group consisting of hydroxylysine, poly(N-methylethylamine), ε-poly-lysine, polyethylenimine, or polyamine, and a borate containing crosslinking agent. The system is formed into a paste through the addition of a variety of materials well known in the art including calcium salts (i.e. phosphates, silicates, sulfates, hydroxides, oxides, borates).

In still a further embodiment, the composition is in the form of a tissue sealant for repair of tissues formed in situ by mixing hyaluronic acid ore derivative thereof with one or more monomer, oligomer, or polymer selected from the group consisting of hydroxylysine, poly(N-methylethylamine), ε-poly-lysine, polyethylenimine, or polyamine and a borate containing crosslinking agent in a metal containing solvent. In situ formation may be accomplished, for example, through the use of a double barrel syringe.

As the term “bioresorbable” is used herein, it is meant that the composition is dissolves and is absorbed by the body. Bioresorbable compositions may dissolve and be absorbed by the body at a rate faster, slower, or at about the same rate as the regeneration of the tissue being treated.

Hyaluronic acid (HA) or derivatives of hyaluronic acid such as hyaluronic acid N-hydroxysuccinimide (HA-NHS) may be used. In addition, the N-hydroxysuccinimide (NHS) alone may be used as an adjunct to form activate esters of carboxylic acids on the HA or other carboxylic acid containing polymers to facilitate crosslinking through a condensation reaction. HA is a bio-polymeric material where repeat unit comprising N-acetyl-D-glucosamine and D-glucuronic acid is linearly repeated in connection. The term ‘HA’ means hyaluronic acid and any of its hyaluronate salts. Hyaluronate salts include but are not limited to inorganic salts such as sodium hyaluronate and potassium hyaluronate etc. and organic salts such as tetrabutylammonium hyaluronate etc.

The molecular weight of HA used may be 1,200-24,000,000, 2,000-3,000, 2,000-5,000, 2,000-10,000, 5,000-10,000, 7,000-12,000, 10,000-15,000. The concentration of HA may be 0.001-10%, 0.001-5%, 0.001-3%, 1.0-3.0%

Suitable polyethylenimines may be linear or branch polymers having a molecular weight of at least 250, preferably with a molecular weight of at least 400, more preferably with a molecular weight of at least 700. The molecular weight of the polyethylenimine should be no greater than 20,000, desirably, no greater than 10,000, more desirably no greater than 5,000, preferably no greater than 3000, and more preferably no greater than 2000. Preferred ranges for the molecular weight of the polyethylenimine component of the composition are from 250 to 20,000, desirably from 400 to 10,000, more desirably from 400 to 3000, and preferably from 700 to 2000.

Suitable polyamines include, but are not limited to polyethylenimine, diethylenetriamine (DETA), triethylenetetramine (TETA), tetraethylenepentamine (TEPA), 1,3 diaminopropane, putrascine, norspermidine, spermidine, homospermidine, thermine, spermine, thermospermine, homospermine, caldopentamine, thermopentamine, homocaldopentamine, caldohexamine, homocaldohexamine, and tetrakis(3-aminopropyl)ammonium.

Borate containing crosslinking agents which may be used include agent capable of crosslinking between the hyaluronic acid groups and the one or more monomer, oligomer, or polymer. For example, boric acid, sodium borate, sodium tetraborate, disodium tetraborate, sodium tetraborate decahydrate, anhydrous borax (Na₂B₄O₇), borax pentahydrate (Na₂B₄O₇.5H₂O), borax decahydrate (Na₂B₄O₇.10H₂O), sodium borohydride, tributylborate, triethanolamine borate, tris(trimethylsilyl)borate, tris-borate-EDTA buffer, triethyl borate, triisopropyl borate, trimethyl borate or an organoborate may be used.

Di- or polyvalent metal ions such as calcium, magnesium, copper, aluminum, strontium, zinc and iron may be used. Some exemplary divalent and polyvalent cations include one or more of Ca²⁺, Cu²⁺, Mg²⁺, Fe²⁺, Fe³⁺, Sr²⁺, Cd²⁺, Al³⁺, Cr²⁺, Co²⁺, Mn²⁺, Ni²⁺, Sn²⁺, and Zn²⁺.

Another embodiment involves a material for use as a medical adhesive or tissue sealant formed in situ from the interaction between Hyaluronic Acid N-Hydroxysuccinimide and a di-, tri- or polyamine such as polyethylenimine, ε-poly-lysine, DETA, TETA, TEPA or other material with two or more primary amine functional groups bound to a single molecule. The materials are provided in a two part system that is mixed at the time of application to the tissue.

The setting time, strength and density of the adhesive/sealant are controlled through the ratio of the functional groups. The materials are packaged into individual luer syringes in a kit or foil pouch and mixed through a syringe connector at the time of use. Another alternative is to package each of the individual components in a double barrel syringe in a kit or foil pouch which is radiation sterilized. The resultant materials are mixed through a static mix tip at the time of use.

### EXAMPLES

The following abbreviations are used in the examples described below:

a. Water: USP Type I Water

b. LYO: Lyophilization (freeze drying)

c. EO: Ethylene Oxide Sterilization

d. HA: Hyaluronic Acid

e. Sodium Tetraborate: Borax

### Example I

Tissue sealant samples were prepared with 1% or 2% Hyaluronic Acid, Boric Acid or Sodium Tetraborate (borax) as the crosslinking agent, varying amounts of crosslinking agent, a differing order of the procedure, and water, Calcium Chloride solution, or bicarbonate buffer. The various combinations of these samples were tested for crosslinking and dissolution. It was determined that samples that contained 1% HA never set, so 2% HA was used. Higher concentrations of crosslinking agents were prepared, and because of the issues that arose from that, the order of procedure was changed. USP Type I water ( ), bicarbonate buffer and 1% CaCl₂ were tested with boric acid and borax. Samples with borax had a consistently higher pH after adding the crosslinking agent and after adding HA. A dissolution study was conducted on borax and boric acid samples with water, bicarbonate buffer, and 1% CaCl₂ solution as the solvent. The boric acid samples set immediately, while the borax samples took longer to set, however, showed more crosslinking over time. The borax samples with the 1% CaCl₂ solution exhibited good mechanical strength and elasticity as compared to the boric acid crosslinked samples.

The initial samples were prepared with polylysine. To determine if polylysine affected the time taken for samples to set, some samples were prepared without polylysine. Additionally, the amount of HA was tested.

TABLE 1
Test Conditions and IDs for Initial Samples

| | | | Crosslinking | |
|---|---|---|---|---|
| | | | agent | |
| | HA | Crosslinking | concentration | Polylysine |
| Sample ID | concentration | agent | (mmol) | (g) |
| AW-01-69-1 | 1% | Borax | 0.6 | 0 |
| AW-01-69-2 | 2% | Borax | 0.6 | 0.2 |
| AW-01-69-3 | 2% | Borax | 1.2 | 0 |
| AW-01-69-4 | 2% | Boric Acid | 1.2 | 0.2 |

To determine if a higher concentration of crosslinking agent was related to more crosslinking, higher concentrations of borax and boric acid were prepared. The higher borax and boric acid did not dissolve in the same amount of water; therefore, the amount of water was increased to from 20 mL to 100 mL for the borax solution. The resultant samples did not set.

TABLE 2
Test Conditions and IDs for more Concentrated Samples

| | | | Crosslinking | |
|---|---|---|---|---|
| | | | agent | |
| | HA | Crosslinking | concentration | Polylysine |
| Sample ID | concentration | agent | (mmol) | (g) |
| AW-01-70-1 | 2% | Borax | 12 | 0.2 |
| AW-01-70-2 | 2% | Borax | 12 | 0 |
| AW-01-72-2 | 2% | Borax | 12 | 0.2 |
| AW-01-72-4 | 2% | Boric Acid | 12 | 0.2 |
| AW-01-72-6 | 2% | Boric Acid | 24 | 0.2 |

Due to these results, the order of procedure was changed. Originally, 100 mL of HA solution was prepared followed by the addition of the crosslinking agent in solution of 20 mL water. The new procedure adds the crosslinking agent to heated water blending and then adding HA.

TABLE 3
Test Conditions and IDs for Samples with Different Solvents

| | | Crosslinking | |
|---|---|---|---|
| | | agent | |
| | Crosslinking | concentration | |
| Sample ID | agent | (mmol) | Solvent |
| AW-01-74-A | Borax | 12 | Water |
| AW-01-74-B | Boric Acid | 12 | Water |
| AW-01-74-C | Boric Acid | 12 | Water |
| AW-01-74-D | Boric Acid | 12 | Buffer |
| AW-01-74-E | Boric Acid | 12 | 1% CaCl₂ |
| AW-01-74-F | Boric Acid | 12 | 2% CaCl₂ |
| AW-01-76-1 | Borax | 12 | Water |
| AW-01-76-2 | Borax | 12 | Buffer |
| AW-01-76-3 | Borax | 12 | 1% CaCl₂ |

The control and test samples were prepared using the methods described in below.
i. Initial samples
   1. 100 mL of water was added to the blender, then 1 or 2 g of HA was added, depending on the desired percentage of HA, and this mixture was blended until homogenous.
   2. If polylysine was used, it was added to the blender at this point, and mixed until homogenous.
   3. The crosslinking agent (borax or boric acid) was added to 20 mL of water and stirred until homogeneous.
   4. The crosslinking solution was then added to the blender and mixed until homogenous.
   5. The final sample was poured into a weighing dish and allowed to crosslink/set.
ii. Samples after Order of Procedure was changed
   1. 100 mL of water was added to a 300 mL beaker and then heated to 50° C.
   2. Crosslinking agent was added and stirred until dissolved.
   3. The solution and HA were added to a blender and blended until homogenous.
   4. If polylysine was used, it was added to the blender at this point, and mixed until homogenous.
   5. The final sample was poured into a weighing dish and left to crosslink and set.
iii. Alternative solvent
   1. 100 mL of the solvent was added to a 300 mL beaker and then heated to 50° C.
      a. 1 g of calcium chloride was mixed until homogenous with 100 mL water.
   2. Crosslinking agent was added and stirred until dissolved.
   3. The solution and HA were added to a blender and mixed until homogenous.
   4. If polylysine was used, it was added to the blender at this point, and blended until homogenous.
   5. The final sample was poured into a weighing dish and allowed to crosslink/set.

The test samples vary by the amount of HA, the solvent, the type of crosslinking agent, the amount of crosslinking agent, the order of procedure, and the presence of polylysine. All samples are evaluated to determine if the formulation sets in less than 10 minutes. All samples were tested for dissolution by filling a weighing dish with water and evaluating the consistency of the material every 2-3 hours. While evaluating for a sample to be set, tests were considered complete when the sample was considered set or if approximately 30 minutes had passed without the sample setting. In a dissolution study, tests were considered complete when the sample was considered dissolved or went multiple days without dissolving. The acceptance criteria for this study are the time taken for the sample to set and the time taken for the sample to dissolve.

Results

Initial Samples

TABLE 4
Test Conditions and Results for Initial Samples

| | | | Crosslinking | | |
|---|---|---|---|---|---|
| | HA | Cross- | Agent | Poly- | |
| | concen- | linking | concentration | lysine | |
| Sample ID | tration | Agent | (mmol) | (g) | Results |
| AW-01- | 1% | Borax | 0.6 | 0 | Did not |
| 69-1 | | | | | set |
| AW-01- | 2% | Borax | 0.6 | 0.2 | Set in <10 |
| 69-2 | | | | | min. |
| AW-01- | 2% | Borax | 1.2 | 0 | Set in <10 |
| 69-3 | | | | | min. |
| AW-01- | 2% | Boric | 1.2 | 0.2 | Set in <10 |
| 69-4 | | Acid | | | min. |

i. Higher Concentration Samples

TABLE 5
Test Conditions and Results for Higher Concentration Samples

| | | | Crosslinking | | |
|---|---|---|---|---|---|
| | HA | Cross- | Agent | Poly- | |
| | concen- | linking | concentration | lysine | |
| Sample ID | tration | Agent | (mmol) | (g) | Results |
| AW-01- | 2% | Borax | 12 | 0.2 | Did not set |
| 70-1 | | | | | |
| AW-01- | 2% | Borax | 12 | 0 | Set in <2 |
| 70-2 | | | | | min. |
| AW-01- | 2% | Borax | 12 | 0.2 | Set in <10 |
| 72-2 | | | | | min. |
| AW-01- | 2% | Boric | 12 | 0.2 | Set in <2 |
| 72-4 | | Acid | | | min. |
| AW-01- | 2% | Boric | 24 | 0.2 | Set in <2 |
| 72-6 | | Acid | | | min. |

Different Solvents

TABLE 6
Test Conditions and pH for Samples with Different Solvents

| | | Crosslinking | | | pH with | |
|---|---|---|---|---|---|---|
| | Cross- | Agent | | | Cross- | pH |
| | linking | concentration | | pH of | linking | with |
| Sample ID | Agent | (mmol) | Solvent | solvent | Agent | HA |
| AW-01- | Borax | 12 | Water | — | — | — |
| 74-A | | | | | | |
| AW-01- | Boric | 12 | Water | — | 3.88 | 5.49 |
| 74-B | Acid | | | | | |
| AW-01- | Boric | 12 | Water | — | — | — |
| 74-C | Acid | | | | | |
| AW-01- | Boric | 12 | Buffer | 9.95 | 8.3 | 8.41 |
| 74-D | Acid | | | | | |
| AW-01- | Boric | 12 | 1% | 9.8 | 5.44 | 5.32 |
| 74-E | Acid | | CaCl₂ | | | |
| AW-01- | Boric | 12 | 2% | 9.99 | 5.55 | 5.39 |
| 74-F | Acid | | CaCl₂ | | | |
| AW-01- | Borax | 12 | Water | — | 9.45 | 9.54 |
| 76-1 | | | | | | |
| AW-01- | Borax | 12 | Buffer | 10.01 | 9.49 | 9.56 |
| 76-2 | | | | | | |
| AW-01- | Borax | 12 | 1% | 9.8 | 8.99 | 8.89 |
| 76-3 | | | CaCl₂ | | | |

TABLE 7
Test Conditions and Dissolution Observations for
Samples with Different Solvents

| | Crosslinking | | | Dissolution |
|---|---|---|---|---|
| Sample ID | Agent | Solvent | Observations | Observations |
| AW-01- | Borax | Water | Did not set | Not tested |
| 74-A | | | | |
| AW-01- | Boric Acid | Water | Set immediately | Dissolved in ~200 min. |
| 74-B | | | | |
| AW-01- | Boric Acid | Water | Set ~15 min. Thinner | Did not set; |
| 74-C | | | consistency than AW-01-74-B | dissolution not |
| | | | | tested |
| AW-01- | Boric Acid | Buffer | Set immediately | Dissolved >2 hrs. |
| 74-D | | | | and <15 hrs. |
| AW-01- | Boric Acid | 1% | Set immediately | Dissolved >1.5 hrs. |
| 74-E | | CaCl₂ | | and <15 hrs. |
| AW-01- | Boric Acid | 2% | Set immediately | Dissolution not |
| 74-F | | CaCl₂ | | tested |
| AW-01- | Borax | Water | Did not set immediately, | Insoluble after 1.5 |
| 76-1 | | | became viscoelastic over time. | hours |
| | | | Sticks to itself as compared to | |
| | | | the weighing dish | |
| AW-01- | Borax | Buffer | Did not set immediately, | Insoluble after 1.5 |
| 76-2 | | | became viscoelastic over time. | hours |
| | | | Sticks to itself as compared to | |
| | | | the weighing dish | |
| AW-01- | Borax | 1% | Did not set immediately, | Insoluble after 1.5 |
| 76-3 | | CaCl₂ | became viscoelastic over time. | hours |
| | | | White. Very stretchy with | |
| | | | similarities to silly putty. Sticks | |
| | | | to itself as compared to the | |
| | | | weighing dish | |

Table 4 indicates that samples employing 2% HA set effectively. Samples were then prepared with 2% HA. Table 5 indicates that samples using 12 and 24 mmol of borax and boric acid, with and without polylysine set within 2 minutes. This did not determine which variables facilitated setting within 2 minutes and additional tests will be conducted. Table 6 indicates that borax will provide more effective crosslinking. Boric acid and borax were tested for dissolution based upon these results.

Table 7 indicates that the boric acid samples generally dissolved between 1.5 hours and 15 hours. The borax samples did not dissolve after 1.5 hours. These results did not conclusively indicate which samples exhibited better dissolution results. The borax samples did not set immediately, while the boric acid samples did. However, the borax samples continued to crosslink over time, while the boric acid samples did not. The borax samples became very elastic and stuck to itself, while boric acid samples stuck to the weigh dishes. Specifically, the borax samples that used 1% CaCl₂ as the solvent exhibited superior elasticity as compared to the other formulations. Over time, these samples crosslinked further than other samples and were selected for future testing. Polylysine did not seem to affect the setting time or the dissolution of any samples.

### Example II

Tissue sealant samples were prepared with 2% Hyaluronic Acid, Sodium Tetraborate (borax) as the crosslinking agent, a 1% Calcium Chloride solution as the solvent, and varying concentrations of borax. The observations on the setting of the sample were taken and a dissolution study was conducted on these samples. Samples had 3 mmol, 6 mmol, 12 mmol, and 24 mmol of borax with and without polylysine. The samples with 3 mmol of borax set in the least amount of time and to the furthest extent. Additionally, the 3 mmol borax samples showed little to no signs of dissolution during a dissolution test. Samples containing a lower concentration of borax of 1.5 mmol were prepared and these underwent dissolution testing. Based on the results, even lower concentrations of borax will be focused on future testing. This study was conducted in order to optimize the formulation of a tissue sealant that sets within ˜10 minutes and does not dissolve in water, based on varying amounts of the crosslinking agent, borax, time for sample to age, and presence of polylysine.

TABLE 8
Test Conditions and IDs for Samples with
Varying Concentrations of Borax

| | Concentration | Concentration |
|---|---|---|
| | of Borax | of polylysine |
| | (mmol/gram of | (mmol/gram of |
| Sample ID | HA) | HA) |
| AW-01-79-A | 3 | 0 |
| AW-01-79-B | 3 | 1.37 |
| AW-01-79-C | 6 | 0 |
| AW-01-79-D | 6 | 1.37 |
| AW-01-79-E | 12 | 0 |
| AW-01-79-F | 12 | 1.37 |
| AW-01-79-G | 24 | 0 |
| AW-01-79-H | 24 | 1.37 |

A portion of the sample was used to test dissolution the same day they were prepared and these samples were labeled with a “−1”.

TABLE 9
Test Conditions and IDs for Samples with Varying Concentrations
of Borax Dissolution 1.5 Hours after Preparation

| | Concentration | Concentration |
|---|---|---|
| | of Borax (per | of polylysine |
| | gram of HA) | (mmol/gram |
| Sample ID | (mmol) | of HA) |
| AW-01-79-A-1 | 3 | 0 |
| AW-01-79-B-1 | 3 | 1.37 |
| AW-01-79-C-1 | 6 | 0 |
| AW-01-79-D-1 | 6 | 1.37 |
| AW-01-79-E-1 | 12 | 0 |
| AW-01-79-F-1 | 12 | 1.37 |
| AW-01-79-G-1 | 24 | 0 |
| AW-01-79-H-1 | 24 | 1.37 |

Samples were aged over 2 days and then a portion of the sample was used to test dissolution. These samples were labeled with a “−2”.

TABLE 10
Test Conditions and IDs for Samples with Varying Concentrations
of Borax Dissolution 2 Days after Preparation

| | Concentration | Concentration |
|---|---|---|
| | of Borax (per | of polylysine |
| | gram of HA) | (mmol/gram |
| Sample ID | (mmol) | of HA) |
| AW-01-79-A-2 | 3 | 0 |
| AW-01-79-B-2 | 3 | 1.37 |
| AW-01-79-C-2 | 6 | 0 |
| AW-01-79-D-2 | 6 | 1.37 |
| AW-01-79-E-2 | 12 | 0 |
| AW-01-79-F-2 | 12 | 1.37 |
| AW-01-79-G-2 | 24 | 0 |
| AW-01-79-H-2 | 24 | 1.37 |

TABLE 11
Test Conditions and IDs for
Samples with 1.5 mmol Borax with Varying Structures

| Sample ID | Composition | Structure |
|---|---|---|
| AW-01-87-1 | without polylysine | thin |
| AW-01-87-2 | without polylysine | thin |
| AW-01-87-3 | without polylysine | thin |
| AW-01-87-4 | without polylysine | none |
| AW-01-87-5 | with 1.37 (mmol/gram | thin |
| | of HA) polylysine | |
| AW-01-87-6 | with 1.37 (mmol/gram | thin |
| | of HA) polylysine | |
| AW-01-87-7 | with 1.37 (mmol/gram | thin |
| | of HA) polylysine | |
| AW-01-87-8 | with 1.37 (mmol/gram | none |
| | of HA) polylysine | |

100 mL of water was used and formed samples sizes of approximately 100 mL in order to be able to test for dissolution. Dissolution was chosen to determine if these formulations could be used as a tissue sealant. All samples were evaluated to determine if that formulation sets. All samples were then tested for dissolution by filling the weigh dish with water and allowed to sit. Samples were evaluated every 2-3 hours.

TABLE 12
Test Conditions and Observations for
Samples with Varying Concentrations

| | Concentration | Concentration | | |
|---|---|---|---|---|
| | of Borax (per | of polylysine | | |
| | gram of HA) | (mmol/gram | Observations just after | Observations 1.5 hours after |
| Sample ID | (mmol) | of HA) | preparing | preparation |
| AW-01- | 3 | 0 | High viscosity | Highest viscosity, does not |
| 79-A | | | | flow, sample impenetrable |
| | | | | with spatula. Sample required |
| | | | | cutting with scissors |
| AW-01- | 3 | 1.37 | High viscosity, not | Highest viscosity, no obvious |
| 79-B | | | different from AW-01- | differences from AW-01-79-A, |
| | | | 79-A | Sample does not flow, |
| | | | | impenetrable with a spatula. |
| | | | | Sample required cutting with |
| | | | | scissors |
| AW-01- | 6 | 0 | Thickest consistency | Highest viscosity which does |
| 79-C | | | | not flow |
| AW-01- | 6 | 1.37 | Thickest consistency, | Highest viscosity which does |
| 79-D | | | not different than AW- | not flow, no obvious |
| | | | 01-79-C | differences from AW-01-79-A. |
| AW-01- | 12 | 0 | Viscosity is thinner | No change |
| 79-E | | | than AW-01-79-AthruD | |
| AW-01- | 12 | 1.37 | Viscosity is thinner | No change |
| 79-F | | | than AW-01-79- | |
| | | | AthruD, no different | |
| | | | than AW-01-79-E | |
| AW-01- | 24 | 0 | Viscosity thinner than | No change |
| 79-G | | | AW-01-79-AthruF | |
| AW-01- | 24 | 1.37 | Viscosity thinner than | No change |
| 79-H | | | AW-01-79-AthruF, no | |
| | | | different than AW-01- | |
| | | | 79-G | |

TABLE 13
Test Conditions and Dissolution Observations for
Samples with Varying Concentrations

| | Con- | | |
|---|---|---|---|
| | centration | | |
| | of | | |
| | Borax | Concentration | |
| | (per gram | of polylysine | |
| | of HA) | (mmol/gram | |
| Sample ID | (mmol) | of HA) | Dissolution Observations |
| AW-01-79- | 3 | 0 | Minimal dissolution, |
| A-1 | | | completely retained shape |
| AW-01-79- | 3 | 1.37 | Minimal dissolution, |
| B-1 | | | completely retained shape |
| AW-01-79- | 6 | 0 | Insoluble, conformed to shape |
| C-1 | | | of the weighing dish |
| AW-01-79- | 6 | 1.37 | Insoluble, conformed to shape |
| D-1 | | | of the weighing dish |
| AW-01-79- | 12 | 0 | Insoluble, conformed to shape |
| E-1 | | | of the weighing dish |
| AW-01-79- | 12 | 1.37 | Insoluble, conformed to shape |
| F-1 | | | of the weighing dish |
| AW-01-79- | 24 | 0 | Highest dissolution, spatula |
| G-1 | | | moves easily through sample |
| AW-01-79- | 24 | 1.37 | Highest dissolution, spatula |
| H-1 | | | moves easily through sample |

TABLE 14
Test Conditions and Observations for
Samples with Varying Concentrations after Aging for 2 Days

| | | Concentration | |
|---|---|---|---|
| | Concentration of | of polylysine | |
| | Borax (per gram | (mmol/gram of | |
| Sample ID | of HA) (mmol) | HA) | Observations |
| AW-01-79- | 3 | 0 | High viscosity, difficult to mix adhered |
| A-2 | | | to the walls of beaker, not flowable |
| AW-01-79- | 3 | 1.37 | High viscosity, did not stick the |
| B-2 | | | beaker, not flowable, easily lifted as |
| | | | one piece |
| AW-01-79- | 6 | 0 | High viscosity, elastic, not flowable |
| C-2 | | | |
| AW-01-79- | 6 | 1.37 | High viscosity, elastic, not flowable |
| D-2 | | | |
| AW-01-79- | 12 | 0 | Flowable, lower viscosity |
| E-2 | | | |
| AW-01-79- | 12 | 1.37 | Flowable, higher viscosity than AW- |
| F-2 | | | 01-79-E-2 |
| AW-01-79- | 24 | 0 | Flowable after being mixed, creamier, |
| G-2 | | | more viscous after 2 days of |
| | | | crosslinking |
| AW-01-79- | 24 | 1.37 | Flowable after being mixed, creamier, |
| H-2 | | | more viscous than AW-01-79-G-2, |
| | | | viscosity further increased after 2 |
| | | | days of crosslinking |

TABLE 15
Test Conditions and Dissolution Observations for
Samples with Varying Concentrations after Aging for 2 Days

| | | Concentration | |
|---|---|---|---|
| | Concentration of | of polylysine | |
| | Borax (per gram | (mmol/gram of | |
| Sample ID | of HA) (mmol) | HA) | Dissolution Observations |
| AW-01-79- | 3 | 0 | Insoluble. Sample remained intact |
| A-2 | | | upon lifting with spatula |
| AW-01-79- | 3 | 1.37 | Insoluble, viscoelastic, adhered to |
| B-2 | | | weighing dish retained shape upon |
| | | | lifting. |
| AW-01-79- | 6 | 0 | Insoluble, viscoelastic. Sample was |
| C-2 | | | not able to be lifted as one piece |
| AW-01-79- | 6 | 1.37 | Insoluble. Performed similarly to |
| D-2 | | | sample AW-01-79-C-2 |
| AW-01-79- | 12 | 0 | Insoluble, friable. Sample broke upon |
| E-2 | | | manipulation with spatula. |
| AW-01-79- | 12 | 1.37 | Insoluble. Performed similarly to |
| F-2 | | | sample AW-01-79-E-2 |
| AW-01-79- | 24 | 0 | Insoluble. Performed similarly to |
| G-2 | | | sample AW-01-79-E-2 |
| AW-01-79- | 24 | 1.37 | Insoluble. Performed similarly to |
| H-2 | | | sample AW-01-79-E-2 |

TABLE 16
Test Conditions and Dissolution Observations for
Samples with 1.5 mmol Borax

| | | | Dissolution after 1 | Dissolution after 1.5 | Dissolution after 2 |
|---|---|---|---|---|---|
| Sample ID | Composition | Immediately | hour | hours | hours |
| AW-01- | without | Softened | Partially | Greater | Complete |
| 87-1 | polylysine | immediately, | dissolved, | dissolution than | dissolution |
| | | absorbing | unable to be | AW-01-87-5thru7 | |
| | | water and | lifted with | could be | |
| | | became | spatula; | manipulated with | |
| | | viscous | | spatula | |
| AW-01- | without | Softened | Exhibited slight | More dissolved | Continued to |
| 87-2 | polylysine | immediately, | dissolution, | than AW-01-87- | exhibit signs of |
| | | absorbing | unable to be | 5thru7, could not | dissolution and |
| | | water and | manipulated | be manipulated | unable to be |
| | | became | with spatula | with spatula | lifted with a |
| | | viscous | | | spatula |
| AW-01- | without | Softened | Exhibited slight | More dissolved | Complete |
| 87-3 | polylysine | immediately, | dissolution, | than AW-01-87- | dissolution |
| | | absorbing | unable to be | 5thru7, could not | |
| | | water and | manipulated | be manipulated | |
| | | became | with spatula | with spatula | |
| | | viscous | | | |
| AW-01- | without | Softened | No change | No change | No change |
| 87-4 | polylysine | immediately, | | | |
| | | absorbing | | | |
| | | water and | | | |
| | | became | | | |
| | | viscous | | | |
| AW-01- | with 1.37 | Softened | Exhibited slight | Able to be lifted | Able to be lifted |
| 87-5 | (mmol/gram | immediately, | dissolution, | with a spatula | with a spatula |
| | of HA) | absorbing | unable to be | continued to | continued to |
| | polylysine | water and | manipulated | exhibit signs of | exhibit signs of |
| | | became | with spatula | dissolution | dissolution |
| | | viscous | | | |
| AW-01- | with 1.37 | Softened | Exhibited slight | Able to be lifted | Able to be lifted |
| 87-6 | (mmol/gram | immediately, | dissolution, | with a spatula | with a spatula |
| | of HA) | absorbing | unable to be | continued to | continued to |
| | polylysine | water and | manipulated | exhibit signs of | exhibit signs of |
| | | became | with spatula; | dissolution | dissolution |
| | | viscous | | | |
| AW-01- | with 1.37 | Softened | Exhibited slight | Able to be lifted | Able to be lifted |
| 87-7 | (mmol/gram | immediately, | dissolution, | with a spatula | with a spatula |
| | of HA) | absorbing | unable to be | continued to | exhibited |
| | polylysine | water and | manipulated | exhibit signs of | significant |
| | | became | with spatula | dissolution | dissolution |
| | | viscous | | | |
| AW-01- | with 1.37 | Softened | No change | No change | No change |
| 87-8 | (mmol/gram | immediately, | | | |
| | of HA) | absorbing | | | |
| | polylysine | water and | | | |
| | | became | | | |
| | | viscous | | | |

NOTE:
These samples did not repair (crosslink back together) after being manipulated by spatula, as observed with previous samples.

The initial observations in Table 6 indicate that the samples prepared with 3 mmol of borax set effectively in the shortest amount of time. Table 14 indicates that samples prepared with 3 mmol of borax displayed superior dissolution results, exhibiting little evidence of dissolution, while other samples dissolved. Table 14 indicates that samples with 3 mmol of borax continued to crosslink as compared to samples with higher concentrations of borax after two days. Table 15 indicates that the dissolution results of all samples improve after two days of samples being allowed to continue crosslinking. Additionally, samples with 3 mmol of borax displayed superior dissolution results, exhibiting no evidence of dissolution. Table 16 indicates that samples that are prepared as thin samples (˜2 mm thick) displayed inferior dissolution results. Most of the thin samples did not exhibit signs of dissolution over 2 hours.

### Example III

Tissue sealant samples were prepared with 2% Hyaluronic Acid, varying amounts of Sodium Tetraborate (borax) below 3 mmol as the crosslinking agent, and 1% Calcium Chloride solution. Samples were prepared with 1.5, 0.5 and 0.1 mmol of borax, with and without polylysine. The setting time of these samples were observed just after preparation and after two days of being allowed to set. The samples were evaluated by adhesion test to collagen substrate. Finally, the samples underwent a dissolution study from 0 minutes to 120 minutes. Based on these observations and tests, the samples containing 1.5 mmol of borax set to the furthest extent in the least amount of time and showed little signs of dissolution in water. All of the samples adhered well to the collagen substrate.

Additionally, tissue sealant samples for a syringe configuration were prepared with varying amounts of Hyaluronic Acid (<2%), 3 mmol of borax as the crosslinking agent, and a 1% CaCl₂ solution. These samples were prepared with 0.2%, 0.5% and 1% of HA solution. Only the samples containing 1% HA became uniform, although still very flowable. After several days of setting, only the 1% HA sample showed changes, crosslinking a considerable amount, while remaining flowable and unsuitable for dissolution testing.

TABLE 17
Test Conditions and Sample IDs for
Varying Concentrations of Borax

| | Concentration of | Concentration of |
|---|---|---|
| | Borax (mmol/gram | polylysine |
| Sample ID | of HA) | (mmol/gram of HA) |
| AW-01- | 1.5 | 0 |
| 93-1 | | |
| AW-01- | 1.5 | 1.37 |
| 93-2 | | |
| AW-01- | 0.5 | 0 |
| 93-3 | | |
| AW-01- | 0.5 | 1.37 |
| 93-4 | | |
| AW-01- | 0.1 | 0 |
| 93-5 | | |
| AW-01- | 0.1 | 1.37 |
| 93-6 | | |

TABLE 18
Test Conditions and Sample IDs for Varying Concentrations of
Hyaluronic Acid

| Sample ID | Percentage of HA |
|---|---|
| AW-01-98-1 | 1 |
| AW-01-98-2 | 0.2 |
| AW-01-98-3 | 0.5 |

Processing Methods
i. 100 mL of water was added to a 300 mL beaker and heated to 45° C.
ii. To prepare a 1% CaCl₂ solution, 1 g of CaCl₂ was mixed until completely dissolved.
iii. The borate containing crosslinking agent was then added and stirred until dissolved (approximately 10 minutes).
iv. The solution with crosslinking agent was mixed with HA in the blender and blended until uniform.
v. If polylysine was used, it was added to the blender at this point, and blended until uniform.
vi. The final sample was poured into a glass beaker and left to set.
vii. AW-01-93-1 through 6 were tested for dissolution by filling the weighing dish with water and allowed time to set. Samples were evaluated approximately every 30 minutes.
viii. AW-01-93-1 through 6 samples were tested for adhesion by cutting 2 inch squares of collagen casing and soaking them in water until hydrated ˜5 minutes. The hydrated collagen casing was lightly dried to remove surface moisture and a small amount of each test article was placed on the collagen substrate. The test article was lifted from the collagen casing to determine if the sample was adhered. The collagen substrate was then lifted from the tissue sealant to determine the extent of adhesion to the test article.

TABLE 19
Observations for Samples with Varying Concentrations of Borax

| | Concentration | Concentration | | |
|---|---|---|---|---|
| | of Borax | of polylysine | | |
| | (mmol per | (mmol per | Observations just after | Observations 2 days after |
| Sample ID | gram of HA) | gram of HA) | preparing | preparation* |
| AW-01- | 1.5 | 0 | More translucent than 3 mmol | Continued to crosslink |
| 93-1 | | | samples. | and was difficult to lift |
| | | | Appeared clearer with | from beaker. Sample |
| | | | more bubbles. Did not | was clearer than 3 mmol |
| | | | exhibit differences from | borax sample. |
| | | | AW-01-93-2. Set in <10 min. | Sample was removed |
| | | | | as one piece. Required |
| | | | | cutting to sample for |
| | | | | dissolution testing. |
| AW-01- | 1.5 | 1.37 | More translucent than 3 mmol | Very similar to AW-01- |
| 93-2 | | | samples. | 93-1, except more |
| | | | Appeared clearer with | bubbles. Sample was |
| | | | more bubbles. Did not | removed as one piece. |
| | | | exhibit differences from | Required cutting to |
| | | | AW-01-93-1. Set in <10 min. | sample for dissolution |
| | | | | testing. |
| AW-01- | 0.5 | 0 | Clearer than AW-01-93- | Appeared crosslinked. |
| 93-3 | | | 1thru2 with more | Clearer than AW-01-93- |
| | | | bubbles. Set in <2 min. | 1thru2. Sample |
| | | | Did not exhibit | removed as one piece. |
| | | | differences from AW-01- | |
| | | | 93-4. Very gel-like, did | |
| | | | not appear crosslinked. | |
| AW-01- | 0.5 | 1.37 | Clearer than AW-01-93- | Appeared more |
| 93-4 | | | 1thru2with more bubbles | crosslinked. Sample |
| | | | as well. Set in <2 min. | was not removed as |
| | | | Did not exhibit | one piece. Sample |
| | | | differences from AW-01- | broke under its own |
| | | | 93-3. Very gel-like and | weight. |
| | | | did not appear highly | |
| | | | crosslinked. | |
| AW-01- | 0.1 | 0 | Very clear with many | Clear with a few |
| 93-5 | | | bubbles, resembled 2% | bubbles. Very gel-like. |
| | | | HA solution. Very gel- | Sample did not move as |
| | | | like. Set in <2 min. Did | a whole, only where the |
| | | | not appear highly | spatula moved through. |
| | | | crosslinked. Did not | Sample was not able to |
| | | | exhibit differences from | be lifted as one piece. |
| | | | AW-01-93-6. | |
| AW-01- | 0.1 | 1.37 | Very clear with many | Very similar to AW-01- |
| 93-6 | | | bubbles, very gel-like. | 93-5, with the exception |
| | | | Set within 2 minutes but | that it contained no |
| | | | did not appear to be | bubbles. |
| | | | crosslinked at all. Did | |
| | | | not show differences | |
| | | | from AW-01-93-5. | |

*Appendix 10.1 contains images of these observations.

TABLE 20
Dissolution Observations for Samples with Varying Concentrations of Borax *

| Time | | | | | | |
|---|---|---|---|---|---|---|
| (min) | AW-01-93-1 | AW-01-93-2 | AW-01-93-3 | AW-01-93-4 | AW-01-93-5 | AW-01-93-6 |
| 0 | Sample was | Sample was | Sample was | Sample was | Sample was | Sample was |
| | lifted as | lifted as | lifted as | not lifted | not lifted | not lifted |
| | one piece | one piece | one piece | as one piece | as one piece | as one piece |
| | | | | and stuck to | | |
| | | | | walls of | | |
| | | | | container | | |
| 15 | Sample was | Sample was | Sample was | Sample was | Sample was | Sample |
| | lifted as | lifted as | lifted as | not completely | not lifted | was lifted |
| | one piece | one piece | one piece | lifted in | in one piece | in one |
| | | | | one piece | | piece |
| 30 | Sample did | Sample did | Sample did | Sample did | Sample did | Sample did |
| | not exhibit | not exhibit | not exhibit | not exhibit | not exhibit | not exhibit |
| | any changes | any changes | any changes | any changes | any changes | any changes |
| 60 | Sample did | Sample did | Sample did | Sample barely | Sample did | Sample did |
| | not exhibit | not exhibit | not exhibit | lifted as | not exhibit | not exhibit |
| | any changes | any changes | any changes | one piece | any changes | any changes |
| 90 | Sample did | Sample did | Sample did | Sample did | Sample did | Sample did |
| | not exhibit | not exhibit | not exhibit | not exhibit | not exhibit | not exhibit |
| | any changes | any changes | any changes | any changes | any changes | any changes |
| 120 | Sample did | Sample did | Sample broke | Sample did | Sample did | Sample did |
| | not exhibit | not exhibit | upon lifting | not exhibit | not exhibit | not exhibit |
| | any changes | any changes | with a spatula | any changes | any changes | any changes |

* Appendix 10.2 contains images of the dissolution of these samples.

TABLE 21
Adhesion Observations for Samples with
Varying Concentrations of Borax

| Sample | | |
|---|---|---|
| ID | Adhesion (+/−) | Observations* |
| AW-01- | + | Sticks very easily to the collagen substrate, the |
| 93-1 | | casing would roll up around the sample |
| AW-01- | + | Sticks very easily to the collagen substrate, the |
| 93-2 | | casing would roll up around the sample |
| AW-01- | + | Sticks very easily to the collagen substrate, the |
| 93-3 | | casing would roll up around the sample |
| AW-01- | + | Sticks it to collagen substrate, spreads across |
| 93-4 | | casing. Sample broke while being held to |
| | | determine if the casing would adhere |
| AW-01- | + | Sticks it to collagen substrate, spreads across |
| 93-5 | | casing. Sample broke while being held to |
| | | determine if the casing would adhere |
| AW-01- | + | Sticks it to collagen substrate, spreads across |
| 93-6 | | casing. Sample broke while being held to |
| | | determine if the casing would adhere |

*Appendix 10.3 contains images containing these observations.

TABLE 22
Observations for Samples with Varying Concentrations of HA

| | Percentage | | |
|---|---|---|---|
| | of | Observations just after | Observations 3 days |
| Sample ID | HA | preparation | after preparation |
| AW-01-98-1 | 1 | Uniform, very | Became |
| | | flowable. After 1 hour, | considerably more |
| | | sample set remaining | crosslinked and was |
| | | very flowable. | still flowable. |
| AW-01-98-2 | 0.2 | Non-uniform with | Appeared as water |
| | | small pieces of HA | with HA gels. |
| | | gels in CaCl₂ solution. | Solution was |
| | | No change after 1 | completely clear. |
| | | hour. | |
| AW-01-98-3 | 0.5 | Uniform, very | Appeared as water. |
| | | flowable. This sample | Slightly more |
| | | was slightly clearer | viscous than AW-01- |
| | | than AW-01-98-1. | 98-2. The solution |
| | | Viscosity increased | was completely |
| | | after 1 hour and | clear. |
| | | remained flowable. | |

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents that are intended to define the spirit and scope of this invention.

1. A composition comprising: (1) hyaluronic acid or derivative thereof, (2) a borate containing crosslinking agent, (3) a di or polyvalent metal ion, and (4) optionally one or more of N-hydroxysuccinimide, a cationic monomer, oligomer, or polymer selected from the group consisting of hydroxylysine, poly(N-methylethylamine), ε-poly-lysine, or polyamine, or a combination thereof.

2. The composition of claim 1, wherein the crosslinking agent is one or more of boric acid, sodium borate, sodium tetraborate, disodium tetraborate, sodium tetraborate decahydrate, anhydrous borax (Na₂B₄O₇), borax pentahydrate (Na₂B₄O₇.5H₂O), borax decahydrate (Na₂B₄O₇.10H₂O), sodium borohydride, tributyl borate, triethanolamine borate, tris(trimethylsilyl)borate, tris-borate-EDTA buffer, triethyl borate, triisopropyl borate, trimethyl borate or an organoborate.

3. The composition of claim 1, wherein the monomer, oligomer, or polymer is ε-poly-lysine.

4. The composition of claim 1, wherein the monomer, oligomer, or polymer is polyethylenimine.

5. The composition of claim 1, wherein the monomer, oligomer, or polymer is diethylenetriamine.

6. The composition of claim 1, wherein the monomer, oligomer, or polymer is triethylenetetramine.

7. The composition of claim 1, wherein hyaluronic acid derivative is in the form of its N-hydroxysuccinimide ester.

8. The composition of claim 1, wherein the monomer, oligomer, or polymer is hydroxylysine

9. The composition of claim 1, wherein the monomer, oligomer or polymer is poly(N-methylethylamine).

10. The composition of claim 1, wherein the di or polyvalent metal ion is calcium, magnesium, copper, aluminum, strontium, zinc or iron.

11. The composition of claim 1, wherein the hyaluronic acid is present at about 0.5% to about 10% by weight of the composition.

12. The composition of claim 1, wherein the hyaluronic acid is present at about 0.5% to about 5% by weight of the composition.

13. The composition of claim 1, wherein the hyaluronic acid is present at about 0.5% to about 2.5% by weight of the composition.

14. The composition of claim 1, wherein the hyaluronic acid is present at about 2% by weight of the composition.

15. The composition of claim 1, wherein the polyamine is one or more of polyethylenimine, diethylenetriamine, triethylenetetramine, tetraethylenepentamine, 1,3 diaminopropane, putrascine, norspermidine, spermidine, homospermidine, thermine, spermine, thermospermine, homospermine, caldopentamine, thermopentamine, homocaldopentamine, caldohexamine, homocaldohexamine, and tetrakis(3-aminopropyl)ammonium.

16. A method for making a bioresorbable tissue repair composition comprising (1) mixing (a) hyaluronic acid or derivative thereof, (b) a borate containing crosslinking agent, (c) a di or polyvalent metal ion, and (d) optionally one or more cationic monomer, oligomer, or polymer selected from the group consisting of hydroxylysine, poly(N-methylethylamine), ε-poly-lysine, polyethylenimine, or polyamine, and (2) lyophilizing or drying the mixture.

17. The method of claim 16, wherein the crosslinking agent is one or more of boric acid, sodium borate, sodium tetraborate, disodium tetraborate, sodium tetraborate decahydrate, anhydrous borax (Na₂B₄O₇), borax pentahydrate (Na₂B₄O₇.5H₂O), borax decahydrate (Na₂B₄O₇.10H₂O), sodium borohydride, tributyl borate, triethanolamine borate, tris(trimethylsilyl)borate, tris-borate-EDTA buffer, triethyl borate, triisopropyl borate, trimethyl borate or an organoborate.

18. The method of claim 16, wherein the monomer, oligomer, or polymer is ε-poly-lysine.

19. The method of claim 16, wherein the monomer, oligomer, or polymer is polyethylenimine.

20. The method of claim 16, wherein the monomer, oligomer, or polymer is diethylenetriamine.

21. The method of claim 16, wherein the monomer, oligomer, or polymer is triethylenetetramine.

22. The method of claim 16, wherein hyaluronic acid derivative is in the form of its N-hydroxysuccinimide ester.

23. The method of claim 16, wherein the monomer, oligomer, or polymer is hydroxylysine

24. The method of claim 16, wherein the monomer, oligomer or polymer is poly(N-methylethylamine).

25. The method of claim 16, wherein the metal containing solution is calcium chloride.

26. The method of claim 16, wherein the hyaluronic acid is present at about 0.5% to about 10% by weight of the composition.

27. The method of claim 16, wherein the hyaluronic acid is present at about 0.5% to about 5% by weight of the composition.

28. The method of claim 16, wherein the hyaluronic acid is present at about 0.5% to about 2.5% by weight of the composition.

29. The method of claim 16, wherein the hyaluronic acid is present at about 2% by weight of the composition.

30. The method of claim 25, wherein the divalent of polyvalent ion solution is a 1% solution of calcium chloride in water.

31. The method of claim 16, further comprising packaging and sterilizing the bioresorbable tissue repair material.

32. A method of repairing tissue comprising contacting tissue in need of treatment thereof with the composition of claim 1.

33. A method of repairing tissue comprising contacting tissue in need of treatment thereof with the composition of claim 1 by mixing the composition in a multi-barrel syringe and applying the composition to the tissue.

34. A method of correcting a tissue defect comprising contacting tissue in need of treatment thereof with the composition of claim 1.

35. A method of correcting a tissue defect comprising contacting tissue in need of treatment thereof with the composition of claim 1 by mixing the composition in a multi-barrel syringe and applying the composition to the tissue.

36. A method of sealing a tissue wound comprising contacting tissue in need of treatment thereof with the composition of claim 1.

37. A method of sealing a tissue wound comprising contacting tissue in need of treatment thereof with the composition of claim 1 by mixing the composition in a multi-barrel syringe and applying the composition to the tissue.

38. A method of connecting tissue comprising contacting tissue in need of treatment thereof with the composition of claim 1.

39. A method of connecting tissue comprising contacting tissue in need of treatment thereof with the composition of claim 1 by mixing the composition in a multi-barrel syringe and applying the composition to the tissue.
